(19) United States
(12) Patent Application Publication
(10) Pub. No.: US 2004/0258639 A1
(43) Pub. Date: Dec. 23, 2004

(54) Decorative skincare product

(75) Inventors: Wiebke Lindemann, Hamburg (DE); Gunhild Hamer, Hamburg (DE); Sven Fey, Norwalk, CT (US)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/809,415

(22) Filed: Mar. 26, 2004

Related U.S. Application Data
(63) Continuation of application No. PCT/EP02/10668, filed on Sep. 23, 2002.

(30) Foreign Application Priority Data
Sep. 28, 2001 (DE) 101 48 264.7

(51) Int. Cl. A61K 7/42; A61K 7/021

(52) U.S. Cl. 424/59; 424/63

(57) ABSTRACT

Cosmetic and/or dermatological preparations comprising

a) silicone oils in a concentration of from 0.1 to 10% by weight

b) fatty alcohols in a concentration of from 0.1 to 15% by weight

c) dicaprylyl ethers in a concentration of from 0.1 to 4% by weight

d) stearic acid/stearate emulsifiers in a concentration of from 0.1 to 10% by weight

e) moisturizers in a concentration of from 0.1 to 15% by weight

f) metal oxide pigments with a particle size of from 5 to 50 μm in a concentration of from 0.1 to 7% by weight

in each case based on the total weight of the preparation,

besides optionally further cosmetic and/or dermatological active ingredients, auxiliaries and additives.

### CROSS-REFERENCE TO RELATED APPLICATIONS

The present application is a continuation of International Application No. PCT/EP02/10668, filed Sep. 23, 2002, the entire disclosure whereof is expressly incorporated by reference herein, which claims priority under 35 U.S.C. § 119 of German Patent Application No. 101 48 264.7, filed Sep. 28, 2001.

The present invention relates to cosmetic and/or dermatological preparations for decorative skin care.

The skin is the largest human organ. Among its many functions (for example for temperature regulation and as a sensory organ) the barrier function, which prevents the skin (and ultimately therefore the entire organism) from drying out, is probably the most important. At the same time, the skin acts as a protective device against the penetration and the absorption of external substances. This barrier function is effected by the epidermis which, as the outermost layer, forms the actual protective sheath against the environment. Providing about one-tenth of the total thickness, it is also the thinnest layer of the skin.

The aim of cosmetic skin care is to strengthen or restore the natural function of the skin as a barrier against environmental influences (e.g. dirt, chemicals, microorganisms) and against the loss of endogenous substances (e.g. water, natural fats, electrolytes). Impairment of this function may lead to increased resorption of toxic or allergenic substances or to attack from microorganisms, resulting in toxic or allergic skin reactions. Another aim of skin care is to compensate for the loss by the skin of lipids and water caused by daily washing. This is particularly important when the natural regeneration ability is insufficient. Furthermore, skincare products should protect against environmental influences, in particular against sun and wind, and delay skin aging.

The trend away from genteel paleness toward “healthy, sporty brown skin” has been unbroken for years for many inhabitants of northern and central Europe. In order to achieve this, people subject their pale skin to solar radiation since this causes pigment formation in the sense of melanin formation. However, the ultraviolet radiation of sunlight has a harmful effect on the skin. Besides the acute damage (sunburn), long-term damage, such as an increased risk of getting skin cancer, arises with excessive irradiation with light from the UVB range (wavelength: 280-320 nm). Moreover, the result of too much UVB and UVA radiation (wavelength: 320-400 nm) is a weakening of the elastic and collagen fibers in connective tissue. This leads to numerous phototoxic and photoallergic reactions, resulting in premature skin aging.

A further problem of sun tanning consists in the uneven brown coloration of the skin. The individual areas of the skin are exposed to sunlight to varying degrees and form different amounts of melanin. In particular, areas of skin which are only rarely exposed to sunlight barely tan. During sun tanning, this therefore leads to an uneven and often unaesthetic tanning of the skin.

By contrast, for many people in sunny regions who have naturally darker skin, a genteel paleness is the beauty ideal. These people generally attempt to protect the skin from solar radiation. Nevertheless, due to varying exposure of the skin to UV radiation, for them too the result is differing degrees of skin pigmentation.

It was therefore the aim of the present invention to develop a skin care product with pleasant sensory properties, in particular with a silky smooth feel on the skin which gives the skin a uniformly healthy appearance and a softly shimmering sheen. The softly shimmering sheen of the skin should be long-lasting and thus not be able to be rubbed off easily, for example by clothing rubbing on the skin.

Surprisingly, the object is achieved by cosmetic and/or dermatological preparations comprising

a) silicone oils in a concentration of from 0.1 to 10% by weight

b) fatty alcohols in a concentration of from 0.1 to 15% by weight

c) dicaprylyl ethers in a concentration of from 0.1 to 4% by weight

d) stearic acid/stearate emulsifiers in a concentration of from 0.1 to 10% by weight

e) moisturizers in a concentration of from 0.1 to 15% by weight

f) metal oxide pigments with a particle size of from 5 to 50 μm in a concentration of from 0.1 to 7% by weight

in each case based on the total weight of the preparation.

Although WO 98/52535 and 99/24001 describe skincare products containing pigments, these specifications were unable to point the way to the present invention since only pigments with a size of from 0.1 to 0.3 μm are used in them. In addition, no iron(III) oxides is used as coloring pigment in these specifications. Although WO 00/24372 discloses particulate constituents in a size of from 2 to 30 μm, for the skincare products disclosed here, vitamin B3 and retinoids are obligatory as constituent for formulating the preparations. All of the comparable skincare products disclosed to date lack attractive sensory properties since, due to their glycerol content, they leave behind a sticky feel on the skin following use.

The composition according to the invention of the lipid phase comprising, inter alia, silicone oils, dicaprylyl ether, fatty alcohols and stearic acid/stearate emulsifiers allows, in combination with metal oxide pigments with a particle size of from 5 to 50 μm, the formulation of coloring skincare products of pleasantly silky smooth feel on the skin and softly shimmering sheen.

According to the invention, the preparation preferably comprises

a) silicone oils in a concentration of from 2 to 4% by weight

b) fatty alcohols in a concentration of from 1 to 7% by weight

c) dicaprylyl ethers in a concentration of from 1 to 4% by weight

d) stearic acid/stearate emulsifiers in a concentration of from 2 to 5% by weight

e) moisturizers in a concentration of from 4 to 10% by weight

f) metal oxide pigments with a particle size of from 5 to 50 μm in a concentration of from 3 to 4% by weight

in each case based on the total weight of the preparation.

The cosmetic and/or dermatological preparations according to the invention comprise dimethicinone and/or cyclomethicone as particularly preferred silicone oils.

The moisturizers according to the invention include, inter alia, polyols, such as glycerol and sorbitol. However, it is also possible to use other compounds, such as ethoxylated polyols and hydrolyzed proteins. In addition, components of the natural moisturizing factor (NMF) of the skin, e.g. urea and certain amino acids, are used.

The cosmetic and/or dermatological preparations according to the invention particularly preferably comprise glycerol as moisturizer.

In addition, it is particularly advantageous according to the invention to use octyl dodecanol and/or myristyl alcohol as fatty alcohol.

The metal oxide pigments preferred according to the invention are titanium dioxide, silicon dioxide, tin oxides and/or iron oxides.

According to the invention, particularly advantageous stearic acid/stearate emulsifiers are stearic acid, glyceryl stearate and sorbitan stearate. Further advantageous coemulsifiers are sorbitan oleate and eucerite.

According to the invention, the preparation according to the invention is advantageously in the form of an emulsion. In this connection, a cosmetic and/or dermatological emulsion according to the invention can preferably additionally comprise one or more water phases besides one or more oil phases and be present, for example, in the form of a W/O (water in oil), W/S (water in silicone oil), O/W (oil in water) or S/W (silicone oil in water) emulsion.

In addition, according to the invention they may also advantageously be in the form of so-called multiple emulsions, such as, for example, W/O/W, O/W/O, W/S/W or S/W/S emulsions. Such formulations may preferably also be a microemulsion (e.g. a PIT emulsion), a solids emulsions (i.e. an emulsion which is stabilized by solids, e.g. a Pickering emulsion), a sprayable emulsion or a hydrodispersion. Furthermore, the preparations for the purposes of the present invention may also be virtually anhydrous (water content less than 5% by weight, based on the total weight of the formulation.

According to the invention, the preparation according to the invention is particularly preferably in the form of a O/W emulsion.

The person skilled in the art is of course aware that high-quality cosmetic compositions are in most cases inconceivable without the customary auxiliaries and additives. The cosmetic preparations according to the invention can therefore comprise cosmetic auxiliaries as are customarily used in such preparations, e.g. preservatives, bactericides, perfume substances, vitamins, antifoams, thickeners, softening substances, fats, oils, waxes or other customary constituents of a cosmetic formulation, such as alcohols, polymers, foam stabilizers, electrolytes or organic solvents.

Corresponding requirements apply mutatis mutandis to the formulation of medicinal preparations.

It is of advantage according to the invention to add self-tanning substances to the cosmetic and/or dermatological preparations according to the invention in a concentration of from 0.1 to 10% by weight, based on the total weight of the formulation. Advantageous self-tanning agents which may be used according to the invention are, inter alia, glycerol aldehyde, hydroxymethylglyoxal, γ-dialdehyde, erythrulose, 6-aldo-D-fructose, ninhydrin, 5-hydroxy-1,4-naphthoquinone (juglone), 2-hydroxy-1,4-naphthoquinone (lawsone). Particularly preferred for the purposes of the invention is 1,3-dihydroxyacetone (DHA).

It is, however, also advantageous according to the invention to add whitening agents to the cosmetic and/or dermatological preparations in a concentration of from 0.1 to 10% by weight, based on the total weight of the formulation. Advantageous whitening agents which may be used are, inter alia, hydroquinones (also in combination with alpha-hydroxy acids), all-trans-retinoic acid, glucocorticoids, dionic acids, lipoic acid, liquorice extract and tyrosine sulfate.

For the purposes of the present invention, the preparation according to the invention can advantageously comprise one or more repellent active ingredients. Advantageous repellent active ingredients for the purposes of the present invention are, for example, N,N-diethyl-3-methylbenzamide, ethyl 3-(N-n-butyl-N-acetylamino)propionate and dimethyl phthalate. Very particular preference is given to the repellent ethyl 3-(N-n-butyl-N-acetylamino)propionate.

Embodiments of the emulsion according to the invention that are advantageous according to the invention comprise one or more repellent active ingredients in a concentration of 1-25% by weight, based on the total weight of the formulation.

Cosmetic and/or dermatological preparations generally comprise a large number of auxiliaries and active ingredients, which may also be used advantageously in the preparations according to the invention.

The customary antioxidants may advantageously be used according to the invention in the preparations which comprise the active ingredient combinations according to the invention.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, 1-carotene, lycopene) and derivatives thereof, aurothioglucose, propylthiouracil and other thiols (e.g. thioredoxine, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and also sulfoximine compounds (e.g. buthionine sulfoximine, homocysteine sulfoximine, buthionine sulfones, penta-, hexa-, heptathionine sulfoximine) in very low tolerated doses (e.g. pmol to μmol/kg), and also (metal) chelating agents (e.g. a-hydroxy-fatty acids, palmitic acid, phytic acid, lactoferrin), α-hydroxy acids (e.g. citric acid, lactic acid, maleic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacetic acid, flavonoids, polyphenols, catechols, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of antioxidants (one or more compounds) in the preparations is preferably 0.001 to 10% by weight, particularly preferably 0.025-2.0% by weight, in particular 0.05-1.0% by weight, based on the total weight of the preparation.

The preparation according to the invention advantageously comprises one or more conditioners. Conditioners preferred according to the invention are, for example, all compounds which are listed in the International Cosmetic Ingredient Dictionary and Handbook (Volume 4, editor: R. C. Pepe, J. A. Wenninger, G. N. McEwen, The Cosmetic, Toiletry, and Fragrance Association, 9th edition, 2002) under section 4 under the keywords Hair Conditioning Agents, Moisturizers, Skin-Conditioning Agents, Skin-Conditioning Agents—Emollient, Skin-Conditioning Agents—Moisturizer, Skin-Conditioning Agents—Miscellaneous, Skin-Conditioning Agents—Occlusive and Skin Protectants, and also all compounds listed in EP 0934956 (p. 11-13) under water soluble conditioning agent and oil soluble conditioning agent. Some of these compounds are listed by name under the constituents of the aqueous phase and of the oil phase. Further conditioners advantageous according to the invention are, for example, the compounds named according to the international nomenclature for cosmetic ingredients (INCI) as Polyquaternium (in particular Polyquaternium-1 to Polyquaternium-56).

Preparations according to the invention for the purposes of the present invention are present, for example, in the form of a cream, a lotion, a cosmetic milk, a mousse cream from an aerosol container and comprise, for example, fats, oils, waxes and/or other fatty substances, and also water and one or more emulsifiers as are customarily used for such a type of formulation.

In particular, the cosmetic and/or dermatological preparations according to the invention are used as body lotion and as face cream, and also as decorative body lotion and/or decorative face cream.

Advantageously, preparations according to the invention can further comprise substances which absorb UV radiation, where the total amount of the filter substances is, for example, 0.1% by weight to 30% by weight, preferably 0.5 to 10% by weight, in particular 1.0 to 6.0% by weight, based on the total weight of the preparations, in order to provide preparations which protect the skin from the entire range of ultraviolet radiation.

If the preparations according to the invention comprise UVB filter substances, these may be oil-soluble or water-soluble. Oil-soluble UVB filters advantageous according to the invention are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethyl-amino)benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropyl benzyl salicylate, homomenthyl salicylate,

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

ester of benzalmalonic acid, preferably di(2-ethylhexyl) 4-methoxybenzal malonate,

2,4,6-trianilino(p-carbo-2′-ethyl-1′-hexyloxy)-1,3,5-triazine.

Advantageous water-soluble UVB filters are, for example:

salts of 2-phenylbenzimidazole-5-sulfonic acid, such as its sodium, potassium or its triethanolammonium salt, and the sulfonic acid itself;

sulfonic acid derivatives of benzophenones, preferably 2-hydroxy-4-methoxy-benzophenone-5-sulfonic acid and its salts;

sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and its salts, and also 1,4-di(2-oxo-10-sulfo-3-bornylidene-methyl)benzene and salts thereof (the corresponding 10-sulfato compounds, for example the corresponding sodium, potassium or triethanolammonium salts), also referred to as benzene-1,4-di(2-oxo-3-bornylidenemethyl-10-sulfonic acid.

The list of specified UVB filters which can be used in combination with the active ingredient combinations according to the invention is not of course intended to be limiting.

It may also be advantageous to use UVA filters which are customarily present in cosmetic preparations. These substances are preferably derivatives of dibenzylmethane, in particular 1-(4′-tert-butylphenyl)-3-(4′-methoxyphenyl)propane-1,3-dione and 1-phenyl-3-(4′-isopropylphenyl)propane-1,3-dione.

In addition, advantageous UVA filters originate from the group of triazines, thus, for example, 2,4-bis{[4-(2-ethylhexyloxy)-2-hydroxy]phenyl}-6-(4-methoxyphenyl)-1,3,5-triazine (trade name Tinosorb® S), and also the group of triazoles, such as, for example, 2,2′-methylenebis[6-2H-benzotriazol-2-yl]4-(1,1,3,3-tetramethylbutyl)phenol) (trade name Tinosorb® M). An advantageous water-soluble UVA filter is 2′-bis-(1,4-phenylene)-1H-benzimidazole-4,6-disulfonic acid sodium salt (trade name Neo Heliopan AP®).

It is possible to use the amounts used for the UVB combination.

The lipid phase of the cosmetic or dermatological emulsions according to the invention can advantageously be chosen from the following substance group:

mineral oils, mineral waxes

oils, such as triglycerides of capric acid or of caprylic acid, and also natural oils, such as, for example, castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates;

silicone oils, such as dimethylpolysiloxanes, diethylpolysiloxanes, diphenyl-polysiloxane and mixed forms thereof.

The oil phase of the emulsions of the present invention is advantageously chosen from the group of esters of saturated and/or unsaturated, branched and/or unbranched alkane carboxylic acids with a chain length of from 3 to 30 carbon atoms and saturated and/or unsaturated, branched and/or unbranched alcohols with a chain length of from 3 to 30 carbon atoms, from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, the group of saturated or unsaturated, branched or unbranched alcohols, and also fatty acid triglycerides, namely triglycerol esters of saturated and/or unsaturated, branched and/or unbranched alkanecarboxylic acids with a chain length of from 8 to 24, in particular 12-18, carbon atoms. The fatty acid triglycerides can, for example, be advantageously chosen from the group of synthetic, semisynthetic and natural oils, e.g. olive oil, sunflower oil, soybean oil, peanut oil, rapeseed oil, almond oil, palm oil, coconut oil, palm kernel oil and the like.

Any mixtures of such oil and wax components are also to be used advantageously for the purposes of the present invention.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic-capric triglyceride, dicaprylyl ether, dicaprylyl carbonate.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase can also advantageously have a content of other cyclic or linear silicone oils than those specified above.

Cyclomethicone (e.g. decamethylcyclopentasiloxane) and dimethicinone is advantageously used as silicone oil to be used according to the invention. However, other silicone oils are also used advantageously for the purposes of the present invention, for example undecamethylcyclotrisiloxane, polydimethylsiloxane, poly(methylphenylsiloxane), cetyldimethicone, behenoxydimethicone.

The aqueous phase of the preparations according to the invention optionally advantageously comprises alcohols, diols or polyols of low carbon number, and ethers thereof, preferably ethanol, isopropanol, propylene glycol, ethylene glycol, ethylene glycol monoethyl or monobutyl ether, propylene glycol monomethyl, monoethyl or monobutyl ether, diethylene glycol monomethyl or monoethyl ether and analogous products. In addition, they may comprise one or more thickeners, which can advantageously be chosen from the group consisting of silicon dioxide, aluminum silicates.

Preparations according to the invention present in the form of emulsions comprise particularly advantageously one or more hydrocolloids. These hydrocolloids may advantageously be chosen from the group of gums, polysaccharides, cellulose derivatives, sheet silicates, polyacrylates and/or other polymers.

Preparations according to the invention present in the form of hydrogels comprise one or more hydrocolloids. These hydrocolloids can advantageously be chosen from the abovementioned group.

The gums include plant and tree saps which harden in air and form resins, or extracts from aquatic plants. From this group, for the purposes of the present invention, gum arabic, carob bean flour, tragacanth, karaya, guar gum, pectin, gellan gum, carrageen, agar, aligns, chondrus, xanthan gum.

Also advantageous is the use of derivatized gums, such as, for example, hydroxypropylguar (Jaguar® HP 8).

The polysaccharides and polysaccharide derivatives include, for example, hyaluronic acid, chitin and chitosan, chondroitin sulfates, starch and starch derivatives.

These cellulose derivatives include, for example, methylcellulose, carboxymethyl-cellulose, hydroxyethylcellulose, hydroxypropylmethylcellulose.

The sheet silicates include naturally occurring and synthetic clay earths, such as, for example, montmorillonite, bentonite, hectorite, laponite, magnesium aluminum silicates such as Veegum®. These can be used as such or in modified form, such as, for example, stearylalkonium hectorites.

In addition, silica gels can also be used advantageously.

The polyacrylates include, for example, Carbopol grades from Goodrich (Carbopol 980, 981, 1382, 5984, 2984, ETD 2001, ETD 2020, ETD 2050 or Pemulen TR1 & TR2).

The polymers include, for example polyacrylamides (Seppigel 305), polyvinyl alcohols, PVP, PVPNA copolymers, polyglycols.

It may be advantageous according to the invention to add further emulsifiers to the preparations according to the invention. These emulsifiers can advantageously be chosen from the group of nonionic, anionic, cationic or amphoteric emulsifiers.

The nonionic emulsifiers include

a) partial fatty acid esters and fatty acid esters of polyhydric alcohols and ethoxylated derivatives thereof (e.g. glyceryl monostearates, sorbitan stearates, glyceryl stearyl citrates, sucrose stearates)

b) ethoxylated fatty alcohols and fatty acids

c) ethoxylated fatty amines, fatty acid amides, fatty acid alkanolamides

d) alkylphenol polyglycol ethers (e.g. Triton X)

e) sugar derivatives (esters and/or ethers of glucose, sucrose and other sugars; e.g. alkyl polyglycosides, such as polyglyceryl-3 methylglucose distearate, methylglucose sesquistearate)

The anionic emulsifiers include

a) soaps (e.g. sodium stearate)

b) fatty alcohol sulfate

c) mono-, di- and trialkylphosphoric esters and ethoxylates thereof.

The cationic emulsifiers include

a) quaternary ammonium compounds with a long-chain aliphatic radical, e.g. distearyldimonium chloride

The amphoteric emulsifiers include

a) alkylamininoalkanecarboxylic acids

b) betaines, sulfobetaines

c) imidazoline derivatives

In addition, there are naturally occurring emulsifiers, which include beeswax, wool wax, lecithin and sterols.

The cosmetic and/or dermatological preparations according to the invention are advantageously used for increasing the bioavailability of α-flavones, in particular α-glycosylrutin, biotin, retinols, ceramides, vitamins and/or ubiquinone, in particular coenzyme Q 10, and/or derivatives thereof which they advantageously comprise according to the invention.

The cosmetic and/or dermatological preparations according to the invention advantageously serve for the treatment and/or prophylaxis of the symptoms of intrinsic and/or extrinsic skin aging, in particular the reduction of wrinkles.

The cosmetic and/or dermatological preparations according to the invention advantageously serve for the treatment and/or prophylaxis of the harmful effects of ultraviolet radiation on the skin.

The cosmetic and/or dermatological preparations according to the invention advantageously serve to improve the elastic properties of the skin, in particular tightening of the skin.

The cosmetic and/or dermatological preparations according to the invention give the skin a pleasantly silky feel on the skin. After rubbing the preparations onto the skin, it has a soft shimmer and becomes silky smooth.

The use of cosmetic and/or dermatological preparations according to the invention for reducing the stickiness of glycerol-containing cosmetic and/or dermatological preparations is in accordance with the invention.

The examples below serve to illustrate the present invention without limiting it. Unless stated otherwise, all amounts, fractions and percentages given are based on the weight and the total amount or on the total weight of the preparations.

| | 1 | 2 | 3 | 4 |
|---|---|---|---|---|
| Stearic acid | 2 | 3 | 2 | 1 |
| Sorbitan oleate | | | | 1 |
| Glyceryl stearate | 3 | 1 | 3 | |
| Sorbitan stearate | | | | 1 |
| Eucerite | | | | 0.1 |
| Cera Microcristallina + | 3 | 2 | | |
| Paraffinum Liquidum | | | | |
| Mineral oil | 3 | 3 | 8 | |
| Glycerol | 8 | 4 | 10 | 5 |
| PEG-150 | | | | 3 |
| Dicaprylyl ether | 1 | 2 | 4 | 3 |
| Myristyl myristate | | 2 | | |
| Isopropyl palmitate | | | | 1 |
| Cetyl palmitate | | | | 1 |
| Myristyl alcohol | 1 | 4 | 1 | 2 |
| Octyldodecanol | 6 | | | 1 |
| Dimethicone | 2 | 2 | 1 | 2 |
| Cyclomethicone | 2 | | 2 | 2 |
| Sodium hydroxide | 0.2 | 0.6 | 0.2 | 0.2 |
| Alcohol denat. | 3 | 3 | 3 | |
| Carbomer | 0.2 | 0.3 | 0.2 | 0.2 |
| Cetyl phosphate | | 0.1 | | |
| Ubiquinone | | | 0.1 | |
| Tocopheryl acetate | | | 0.5 | 0.5 |
| Cl 77891 + Mica | 3 | 3 | 2 | 3 |
| Mica + Cl 77891 + | 1 | 1 | 2 | |
| Cl 77491 | | | | |
| Phenoxyethanol | | 0.3 | | 0.5 |
| Ethylparaben | 0.2 | | 0.2 | 0.1 |
| Methylparaben | 0.3 | 0.1 | 0.3 | 0.1 |
| Propylparaben | 0.1 | | 0.1 | |
| Polyglyceryl-2 caprate | | 0.4 | | |
| Perfume | 0.3 | 0.2 | 0.3 | 0.2 |
| Water | ad 100 | ad 100 | ad 100 | ad 100 |

1.-9. (canceled)

10. A cosmetic or dermatological composition comprising:
(a) from 0.1% to 10% by weight of a silicone oil;
(b) from 0.1% to 15% by weight of a fatty alcohol;
(c) from 0.1% to 4% by weight of a dicaprylyl ether;
(d) from 0.1% to 10% by weight of an emulsifier selected from one or more or stearic acid and stearates;
(e) from 0.1% to 15% by weight of a moisturizer; and
(f) from 0.1% to 7% by weight of a metal oxide pigment having a particle size of from 5 to 50 μm.

11. The composition of claim 10, wherein the silicone oil comprises at least one of dimethicone and cyclomethicone.

12. The composition of claim 11, wherein the composition comprises from 2% to 4% by weight of the silicone oil.

13. The composition of claim 10, wherein the fatty alcohol comprises at least one of octyldodecanol and myristyl alcohol.

14. The composition of claim 13, wherein the composition comprises from 1% to 7% by weight of the fatty alcohol.

15. The composition of claim 10, wherein the emulsifier comprises at least one of stearic acid, glyceryl stearate and sorbitan stearate.

16. The composition of claim 15, wherein the composition comprises from 2% to 5% by weight of the emulsifier.

17. The composition of claim 10, wherein the composition further comprises a coemulsifier.

18. The composition of claim 17, wherein the coemulsifier comprises at least one of sorbitan oleate and eucerite.

19. The composition of claim 10, wherein the moisturizer comprises glycerol.

20. The composition of claim 19, wherein the composition comprises from 4% to 10% by weight of the moisturizer.

21. The composition of claim 10, wherein the metal oxide pigment comprises at least one of titanium dioxide, silicon dioxide, tin oxides and iron oxides.

22. The composition of claim 21, wherein the composition comprises from 3% to 4% by weight of the metal oxide pigment.

23. The composition of claim 10, wherein the composition comprises at least one of
(a) from 2% to 4% by weight of the silicone oil;
(b) from 1 to 7% by weight of the fatty alcohol;
(c) from 1% to 4% by weight of the dicaprylyl ether;
(d) from 2% to 5% by weight of the emulsifier;
(e) from 4% to 10% by weight of the moisturizer; and
(f) from 3% to 4% by weight of the metal oxide pigment.

24. The composition of claim 23, wherein the silicone oil comprises at least one of dimethicone and cyclomethicone.

25. The composition of claim 23, wherein the fatty alcohol comprises at least one of octyldodecanol and myristyl alcohol.

26. The composition of claim 23, wherein the emulsifier comprises at least one of stearic acid, glyceryl stearate and sorbitan stearate.

27. The composition of claim 26, wherein the composition further comprises at least one of sorbitan oleate and eucerite.

28. The composition of claim 23, wherein the moisturizer comprises glycerol.

29. The composition of claim 23, wherein the metal oxide pigment comprises at least one of titanium dioxide, silicon dioxide, tin oxides and iron oxides.

30. The composition of claim 10, wherein the composition further comprises a self-tanning substance.

31. The composition of claim 30, wherein the self-tanning substance comprises 1,3-dihydroxyacetone.

32. The composition of claim 10, wherein the composition further comprises from 0.1% to 10% by weight of at least one skin whitening agent.

33. The composition of claim 10, wherein the composition further comprises from 1% to 25% by weight of at least one repellent agent.

34. The composition of claim 33, wherein the repellent agent comprises ethyl 3-(N-n-butyl-N-acetylamino)propionate.

35. A cosmetic or dermatological composition comprising:
(a) from 2% to 4% by weight of a silicone oil which comprises at least one of dimethicone and cyclomethicone;
(b) from 1 to 7% by weight of a fatty alcohol which comprises at least one of octyldodecanol and myristyl alcohol;
(c) from 1% to 4% by weight of a dicaprylyl ether;
(d) from 2% to 5% by weight of an emulsifier which comprises at least one of stearic acid, glyceryl stearate and sorbitan stearate;
(e) from 4% to 10% by weight of a moisturizer which comprises glycerol; and
(f) from 3% to 4% by weight of a metal oxide pigment having a particle size of from 5 to 50 μm and comprising at least one of titanium dioxide, silicon dioxide, tin oxides and iron oxides.

36. An emulsion which comprises the composition of claim 10.

37. The emulsion of claim 36, wherein the emulsion comprises an O/W emulsion.

38. A body lotion which comprises the composition of claim 10.

39. A face cream which comprises the composition of claim 10.

40. A method of reducing the stickiness of a glycerol-containing cosmetic or dermatological composition, wherein the method comprises incorporating the glycerol in a composition which comprises
(a) from 0.1% to 10% by weight of a silicone oil;
(b) from 0.1% to 15% by weight of a fatty alcohol;
(c) from 0.1% to 4% by weight of a dicaprylyl ether;
(d) from 0.1% to 10% by weight of an emulsifier selected from one or more or stearic acid and stearates;
(e) from 0.1% to 15% by weight of a moisturizer which comprises the glycerol; and
(f) from 0.1% to 7% by weight of a metal oxide pigment having a particle size of from 5 to 50 μm.

41. The method of claim 40, wherein the composition comprises
(a) from 2% to 4% by weight of the silicone oil;
(b) from 1 to 7% by weight of the fatty alcohol;
(c) from 1% to 4% by weight of the dicaprylyl ether;
(d) from 2% to 5% by weight of the emulsifier;
(e) from 4% to 10% by weight of the moisturizer; and
(f) from 3% to 4% by weight of the metal oxide pigment.
